(19) United States
(12) Patent Application Publication
(10) Pub. No.: US 2008/0219975 A1
(43) Pub. Date: Sep. 11, 2008

(54) VEGFR3 INHIBITORS

(76) Inventors: Timothy Pietro Suren PERERA, Geel (BE); Matthias Luc A. Versele, Leuven (BE); Martin John Page, Schilde (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/875,288

(22) Filed: Oct. 19, 2007

Related U.S. Application Data
(60) Provisional application No. 60/863,198, filed on Oct. 27, 2006.
(60) Provisional application No. 60/976,210, filed on Sep. 28, 2007.

(51) Int. Cl. A61K 39/395; A61K 31/519; A61P 35/00; A61K 31/704

(52) U.S. Cl. 424/133.1; 514/257; 514/34; 424/158.1

(57) ABSTRACT

The present invention relates to the use of some of the macrocyclic quinazoline derivatives described in PCT publication WO2004/105765 as inhibitors of VEGFR3 mediated biological activities, especially those activities which are mediated by VEGFR3 ligands VEGF-C and/or VEGF-D.

### CROSS-REFERENCE TO RELATED APPLICATION

This application claims priority to U.S. Provisional Application for Patent No. 60/863,198, filed Oct. 27, 2006, and U.S. Provisional Application for Patent No. 60/976,210, filed Sep. 28, 2007, the entire disclosures of which are hereby incorporated in their entirely.

### FIELD OF THE INVENTION

The present invention is concerned with the finding that some of the macrocyclic quinazoline derivatives described in PCT publication WO2004/105765 are useful as inhibitors of VEGFR3 mediated biological activities, especially those activities which are mediated by VEGFR3 ligands VEGF-C and/or VEGF-D.

### BACKGROUND OF THE INVENTION

Cancer is still a major cause of death in the world at the beginning of the 21st century and remains a major focus for ongoing research and development.

In recent years a promising approach to the therapeutic intervention of cancer has focused on antiangiogenesis therapies. This approach to intervening in cancer progression takes advantage of the idea that inhibiting or otherwise limiting the blood supply to tumors will deplete the tumor of oxygen and nutrients and will cause arrest of tumor cell growth and proliferation. This approach has been found to be effective and there are presently over 20 anti-angiogenic drugs undergoing various stages of evaluation in phase I, II or III clinical trials and numerous others in preclinical development.

While there are many different forms of cancer exhibiting a wide variety of properties, one factor which many cancers share is that, in order to be fatal, they must metastasize. Until such time as metastasis occurs, a tumor, although it may be malignant, is confined to one area of the body. This may cause discomfort and/or pain, or even lead to more serious problems, nevertheless if it can be located prior to metastasis, the cancer may be managed or even removed by surgical intervention. So long as the residual cancer cells are kept in check, such a discrete cancer may be controlled without significant problems. However, metastasis will cause the cancerous cells to invade the body and while surgical resection may remove the primary tumor, the metastatic spread of the cancer to disparate sites is very difficult to manage.

Metastasis to regional lymph nodes via lymphatic vessels is a common step in the progression of cancer. Metastasis is an important prognostic factor in many types of cancer and forms the basis for surgical and radiation treatment of local lymph nodes. The process of tumor metastasis is a multistage event involving local invasion and destruction of intercellular matrix, intravasation into blood vessels, lymphatic or other channels of transport, survival in the circulation, extravasation out of the vessels in the secondary site and growth in the new location (Idler, et al., Adv. Cancer Res. 28, 149-250 (1978), Liotta, et al., Cancer Treatment Res. 40, 223-238 (1988), Nicolson, Biochim. Biophy. Acta 948, 175-224(1988) and Zetter, N. Eng. J. Med. 322,605-612 (1990)). Success in establishing metastatic deposits requires tumor cells to be able to accomplish these steps sequentially.

Recently, several lines of evidence have indicated that lymphangiogenesis, the formation of lymphatic vessels, promotes lymphatic metastasis (Stacker et al., Nature Med. 7(2), 186-191 (2001); Skobe et al., Nature Med. 7(2), 192-8 (2001); Makinen et al., Nature Med. 7(2), 199-205 (2001)). The control of lymphangiogenesis may provide a new strategy for preventing lymph node metastasis in cancer therapy.

Recent studies have shown that a member of the vascular endothelial growth factor (VEGF) family, VEGF-C, stimulates lymphangiogenesis and lymphatic endothelial cell growth and migration upon binding to its receptor, VEGFR3 (Karkkainen M J, et al, Semin Cell Dev Biol. 13:9-18 (2002)). VEGF-C has also been shown to promote lymphatic-mediated metastasis via induction of tumor-associated lymphangiogenesis in numerous solid cancers, such as gastric cancer, prostatic cancer, human colorectal cancer, invasive cervical cancer, breast cancer metastases and human melanoma metastases. In support of these are preclinical data demonstrating that VEGF-C overexpression in cancer cells significantly increases tumor-associated lymphangiogenesis, resulting in enhanced metastasis to regional lymph nodes (Stacker S A., et al, FASEB J 16:922-34 (2002)). In addition, blockade of VEGF-C/D-mediated signaling has been shown to suppress tumor lymphangiogenesis and lymph node metastases in mice (He Y., et al., J Natl Cancer Inst. 94:819-25 (2002)).

VEGFR3, a transmembrane tyrosine kinase receptor is expressed broadly in endothelial cells during early embryogenesis (Pajusola K., et al, Cancer Res. 53(16):3845 (1992)). During later stages of development, the expression of VEGFR-3 becomes restricted to developing lymphatic vessels [Kaipainen, A., et al., Proc. Natl. Acad. Sci. USA, 92: 3566-3570 (1995)]. In adults, the lymphatic endothelium and some high endothelial venules express VEGFR-3, and increased expression occurs in lymphatic sinuses in metastatic lymph nodes and in lymphangioma. VEGFR-3 is also expressed in a subset of CD34+ hematopoietic cells which may mediate the myelopoietic activity of VEGF-C demonstrated by overexpression studies. Targeted disruption of the VEGFR-3 gene in mouse embryos leads to failure of the remodeling of the primary vascular network, and death after embryonic day 9.5 [Dumont et al., Science, 282: 946-949 (1998)]. These studies suggest an essential role for VEGFR-3 in the development of the embryonic vasculature, and also during lymphangiogenesis.

From the foregoing, it will be apparent that inhibitors of VEGFR3, have tremendous potential as therapeutics, and new agents of this type represent a continuing need in the art for inhibiting growth and/or spread of a variety of neoplastic disorders or cell proliferative disorders. Inhibition of VEGFR3 activity, including inhibition of ligand binding to VEGFR3 is useful in the treatment of mammalian subjects that have been diagnosed with a disease characterized by proliferation of endothelial cells that express VEGFR-3. For example, many tumors are characterized by blood vessel or lymphatic vessel neovascularization, wherein the neovascularization comprises endothelial cells that express VEGFR-3. In some cancers, the cancer cells themselves express VEGFR3, and represent the target cells. In another, possibly overlapping set of cancers, the cancer cells express a VEGFR-3 ligand selected from VEGF-C and VEGF-D. The ligand is believed to be involved in recruiting endothelial cells and stimulating their growth, thereby facilitating nourishment and/or spread of the cancer.

### SUMMARY OF THE INVENTION

The invention is directed in part to methods of treating or preventing VEGFR3 mediated biological activities utilizing certain compounds described in WO2004/105765, the disclosure of which is hereby incorporated by reference in its entirety.

In a related embodiment, the invention provides a method of inhibiting metastatic spread of a cancer in a mammalian subject comprising administering to a mammalian subject suspected of having cancer a compound of the invention, in an amount effective to inhibit metastatic spread of the cancer; and a method for treating cancer comprising administering to a mammalian subject diagnosed with a cancer a composition comprising a compound of the invention, in an amount effect to reduce growth or neoplastic spread of the cancer. It will be appreciated that any reduction in the rate of cancer growth or spread (which can prolong life and quality of life) is indicative of successful treatment. In preferred embodiments, cancer growth is halted completely. In still more preferred embodiments, cancers shrink or are eradicated entirely. Preferred subjects for treatment are human subjects, but other animals, especially murine, rat, canine, bovine, porcine, primate, and other model systems for cancer treatment, are contemplated.

In some cancers that express VEGFR-3, direct inhibition of cancer growth or cancer killing may be the mechanism. Treatment of all cancers that express VEGFR-3 and all cancers characterized by angiogenesis or lymphangiogenesis in and around a growing tumor is contemplated. For example, treatment is contemplated of cancers of a tissue, organ, or cell selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood. In a particular embodiment of the present invention, treatment is contemplated of cancers of a tissue, organ or cell selected from the group consisting of Breast, Lung, Prostate and Colon.

In one variation of the foregoing methods of treatment, the compounds are administered along with a second cancer therapeutic agent. The second agent can be any chemotherapeutic agent, radioactive agent, radiation, nucleic acid encoding a cancer therapeutic agent, antibody, protein, and/or other anti-lymphangiogenic agent or an anti-angiogenic agent. The second agent may be administered before, after, or concurrently with the compounds of the invention.

In one variation, the subject to be treated has been diagnosed with an operable tumor, and the administering step is performed before, during, or after the tumor is resected from the subject. Compound treatment in conjunction with tumor resection is intended to reduce or eliminate regrowth of tumors from cancer cells that fail to be resected.

Stated more generically, the invention provides a method of treating a pathology characterized by VEGFR-3 binding to a natural ligand that binds VEGFR-3, comprising the step of administering to an individual in need thereof a compound of the invention.

### BRIEF DESCRIPTION OF THE DRAWINGS

FIG. 1: Effect of 4,6-ethanediylidenepyrimido [4,5-b][6,1,12]benzoxadiazacyclopentadecine, 17-bromo-8,9,10,11,12,13,14,19-octahydro-20-methoxy-13-methyl- (Compound 2) on VEGF-C induced VEGFR3 activity. Top frame provides the results of VEGFR-3 immunoprecipitates transferred to a nitrocellulose membrane, stained with phospho-tyrosine antibodies. Lower frame provides the results of VEGFR-3 immunoprecipitates transferred to a nitrocellulose membrane, stained with VEGFR-3 antibodies. Left lanes 1 and 2 provide the negative and positive controls, i.e. DMSO treatment in the absence and presence of VEGF-C.

FIG. 2: VEGF and VEGF-C stimulation of Erk1/2 phosphorylation in HMVECd cells in the presence of 5 uM of Compound 2. Top lane provides staining of phoshorylated Erk 1/2 (P-Erk 1/2) using Thr202/Tyr204 mouse monoclonal antibody as primary antibody and goat anti-mouse conjugated to IRDye 800 nm as secondary antibody. Bottom lane provides staining of total Erk 1/2 using Erk 1/2 specific rabbit polyclonal antibody as primary antibody and goat anti-rabbit conjugated to Alexa 680 nm as secondary antibody. Left panel provides the results for VEGF induced phosphorylation of Erk-1/2 in HMVECd cells. Right panel provides the results for VEGF-C induced phosphorylation of Erk 1/2 in HMVECd cells.

### DETAILED DESCRIPTION OF THE INVENTION

WO-2004/105765 describes the preparation, formulation and pharmaceutical properties of a class of macrocyclic quinazoline derivatives of formula (I) as multi targeted kinase inhibitors.

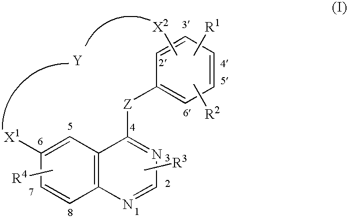

It has now been found that a particular group of compounds within the aforementioned class have VEGFR3 inhibitory activity, that makes them useful in the treatment or prevention of VEGFR3 mediated biological activities, in particular for the treatment or prevention of metastatic spread of a cancer in a mammalian subject.

Accordingly, in one aspect the present invention provides the use of the compounds of formula (I) wherein;
Z represents NH;
Y represents —C₃₋₉alkyl-, —C₁₋₅alkyl-NR¹³—C₁₋₅alkyl-, —C₁₋₅alkyl-NR¹⁴—CO—C₁₋₅alkyl-, —C₁₋₃alkyl-NH—CO-Het²⁰-, or -Het²²—CH₂—CO—NH-C₁₋₃alkyl-;
X¹ represents O, or —O—C₁₋₂alkyl-;
X² represents a direct bond, —C₁₋₂alkyl-, O, —O—C₁₋₂alkyl-, NR¹² or NR¹²-C₁₋₂alkyl-;
R¹ represents hydrogen, cyano, halo or hydroxy, preferably halo;
R² represents hydrogen, cyano, halo, or hydroxy;
R³ represents hydrogen;
R⁴ represents C₁₋₄alkyloxy-;
R¹² represents hydrogen, or C₁₋₄alkyl-;
R¹³ represents hydrogen or C₁₋₄alkyl;
R¹⁴ represents hydrogen or C₁₋₄alkyl;
Het²⁰ represents pyrrolidinyl, piperazinyl or piperidinyl; and
Het²² represents pyrrolidinyl, piperazinyl or piperidinyl;
the pharmaceutically acceptable acid or base addition salts and the stereochemically isomeric forms thereof, in the manufacture of a medicament for the treatment or prevention of VEGFR3 mediated biological activities, such as for example in the treatment or prevention of metastatic spread of a cancer in a mammalian subject.

A further aspect of the present invention is directed to a method for the treatment or prevention of a VEGFR3 mediated biological activity, such as for example in the treatment or prevention of metastatic spread of a cancer in a mammalian subject, comprising administering therapeutically effective amount of a compound of

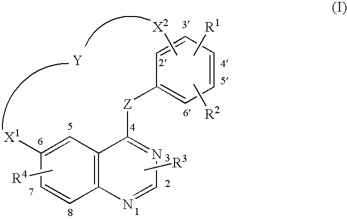

wherein;
Z represents NH;
Y represents —C₃₋₉alkyl-, —C₁₋₅alkyl-NR¹³—C₁₋₅alkyl-, —C₁₋₅alkyl-NR¹⁴—CO—C₁₋₅alkyl-, —C₁₋₃alkyl-NH—CO-Het²⁰-, or -Het²²-CH₂—CO—NH—C₁₋₃alkyl-;
X¹ represents O, or —O—C₁₋₂alkyl-;
X² represents a direct bond, —C₁₋₂alkyl-, O, —O—C₁₋₂alkyl-, NR¹² or NR¹²—C₁₋₂alkyl-;
R¹ represents hydrogen, cyano, halo or hydroxy, preferably halo;
R² represents hydrogen, cyano, halo, or hydroxy;
R³ represents hydrogen;
R⁴ represents C₁₋₄alkyloxy-;
R¹² represents hydrogen, or C₁₋₄alkyl-;
R¹³ represents hydrogen or C₁₋₄alkyl;
R¹⁴ represents hydrogen or C₁₋₄alkyl;
Het²⁰ represents pyrrolidinyl, piperazinyl or piperidinyl;
Het²² represents pyrrolidinyl, piperazinyl or piperidinyl; and
the pharmaceutically acceptable acid or base addition salts and the stereochemically isomeric forms thereof:
to a mammalian subject in need of such treatment.

Further VEGFR3 mediated biological activities are meant to include;
metastatic spread of a cancer in a mammalian subject. Preferred subjects for treatment are human subjects, but other animals, especially murine, rat, canine, bovine, porcine, primate and other model systems for cancer treatment are contemplated.
Metastasis to regional lymph nodes via lymphatic vessels. It accordingly provides the use of the compounds according to the invention in the treatment or prevention of lymph node metastasis;
tumor-associated lymphangiogenesis in cancers, such as for example in gastric cancer, prostatic cancer, human colorectal cancer, invasive cervical cancer, breast cancer, Kapsoi's sarcoma and melanoma. It accordingly provides the use of the compounds according to the invention in the treatment or prevention of tumor-associated lymphangiogenesis in cancers, such as for example in gastric cancer, prostatic cancer, human colorectal cancer, invasive cervical cancer, breast cancer, Kapsoi's sarcoma and melanoma;
recruitment and proliferation of endothelial cells that express VEGFR3 in neovascularization of cancer cells that express a VEGFR3 ligand selected from VEGF-C or VEGF-D. It accordingly provides the use of the compounds according to the invention in the treatment of cancers characterized by angiogenesis or lymphangiogenesis in and around a growing tumor.

In a further embodiment, the present invention provides the use of an aforementioned compound of formula (I) for the preparation of a pharmaceutical composition for treatment of cancers of a tissue, organ or cell selected from the group consisting of Breast, Lung (including the treatment of both small cell and non-small cell lung cancer), Colon and Prostate.

The present invention also concerns a method of treating breast cancer, lung cancer, colon cancer and/or prostate cancer in a mammal, comprising the step of administering a therapeutically effective amount of an aforementioned compound of formula (I) to said mammal.

In an even further embodiment, the present invention provides the use of an aforementioned compound of formula (I) for the preparation of a pharmaceutical composition for treating advanced breast cancer. The term “advanced breast cancer” is used herein to denote breast cancer which has not responded to previous treatment, or which has recurred following such treatment, and also breast cancer in patients who present with metastatic disease at diagnosis.

The present invention also concerns a method of treating advanced breast cancer in a mammal, particularly a woman, comprising the step of administering a therapeutically effective amount of an aforementioned compound of formula (I) to said mammal.

In particular, the present invention is concerned with the use of those compounds of formula (I) wherein one or more of the following restrictions apply;
Z represents NH;
Y represents —C₃₋₉alkyl-, —C₁₋₅alkyl-NR¹⁴—CO—C₁₋₅alkyl-, or —C₁₋₃alkyl-NH—CO-Het²⁰-;
X¹ represents O;
X² represents —C₁₋₂alkyl-, O, or NR¹²—C₁₋₂alkyl-;
R¹ represents hydrogen or halo; in particular R¹ represents hydrogen or chloro; more in particular R¹ represents hydrogen;
R² represents hydrogen or halo; in particular R² represents hydrogen, chloro or bromo; more in particular R² represents chloro or bromo;
R³ represents hydrogen;
R⁴ represents C₁₋₄alkoxy; in particular R⁴ represents methoxy;
R¹² represents C₁₋₄alkyl-; in particular R¹² represents methyl;
R¹⁴ represents hydrogen or C₁₋₄alkyl; in particular R¹⁴ represents hydrogen or methyl; more in particular R¹⁴ represents hydrogen;
Het²⁰ represents pyrrolidinyl, piperazinyl or piperidinyl; in particular Het²⁰ represents piperidinyl.

Also of interest in the aforementioned uses, are those compounds of formula (I) wherein R¹ is at position 3′, R² is at position 5′, R⁴ is at position 7 and X² is at position 2′, using the numbering as presented in Formula (I) above.

Most preferred compounds are those compounds selected from the group consisting of;
4,6-ethanediylidene-19H-pyrimido[4,5-b][6,13,1]benzodioxaazacyclo-pentadecine, 15-chloro-8,9,10,11,12,13-hexahydro-20-methoxy-;
12H-4,6-ethanediylidene-13,17-methanopyrimido[4,5-b][6,1,10,16]benzoxatriazacyclononadecin-12-one, 21-chloro-8,9,10,11,13,14,15,16,18,23-decahydro-25-methoxy-;
4,6-ethanediylidene-12H-pyrimido[4,5-b][6,1,10,13]benzoxatriazacyclohexadecin-12-one, 18-chloro-8,9,10,11,13,14,15,20-octahydro-21-methoxy-13,14-dimethyl-; and
4,6-ethanediylidenepyrimido[4,5-b][6,1,12]benzoxadiazacyclopentadecine, 17-bromo-8,9,10,11,12,13,14,19-octahydro-20-methoxy-13-methyl-; or a pharmaceutically acceptable acid addition salt thereof. The latter compound is especially preferred.

Most preferred compounds for use in accordance with the present invention are those selected from the group consisting of compounds having the following structure:

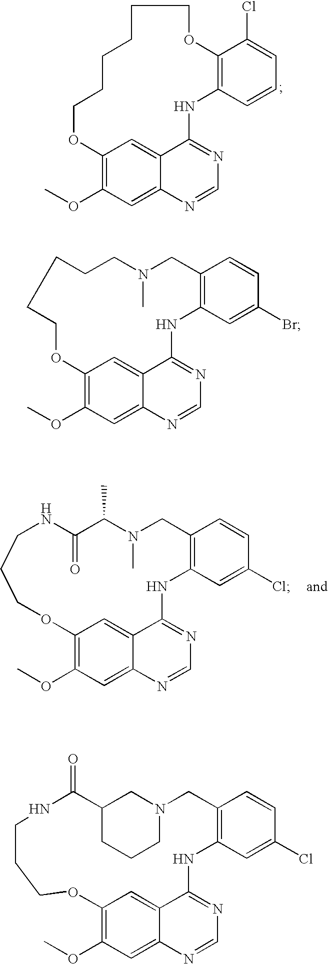

; or a pharmaceutically acceptable acid addition salt thereof.

The following compound is especially preferred:

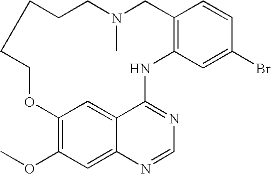

or a pharmaceutically acceptable acid addition salt thereof.

As used in the foregoing definitions and hereinafter,
halo is generic to fluoro, chloro, bromo and iodo;
C₁₋₂alkyl defines methyl or ethyl;
C₁₋₃alkyl defines straight and branched chain saturated hydrocarbon radicals having from 1 to 3 carbon atoms such as, for example, methyl, ethyl, propyl and the like;
C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like;
C₁₋₅alkyl defines straight and branched chain saturated hydrocarbon radicals having from 1 to 5 carbon atoms such as, for example, methyl, ethyl, propyl, butyl, pentyl, 1-methylbutyl, 2,2-dimethylpropyl, 2,2-dimethylethyl and the like;
C₃₋₉alkyl defines straight and branched chain saturated hydrocarbon radicals having from 3 to 9 carbon atoms such as propyl, butyl, pentyl, hexyl, heptyl, octyl, nonyl and the like;
C₁₋₄alkyloxy defines straight or branched saturated hydrocarbon radicals such as methoxy, ethoxy, propyloxy, butyloxy, 1-methylethyloxy, 2-methylpropyloxy and the like;
the term “CO” refers to a carbonyl group.

The pharmaceutically acceptable acid or base addition salts as mentioned hereinabove are meant to comprise the therapeutically active non-toxic acid and non-toxic base addition salt forms which the compounds of formula (I) are able to form. The compounds of formula (I) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds of formula (I) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formula (I) are able to form. Examples of such forms are e.g. hydrates, alcoholates and the like.

The term stereochemically isomeric forms of compounds of formula (I), as used hereinbefore, defines all possible compounds made up of the same atoms bonded by the same sequence of bonds but having different three-dimensional structures which are not interchangeable, which the compounds of formula (I) may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of formula (I) both in pure form or in admixture with each other are intended to be embraced within the scope of the present invention.

Some of the compounds of formula (I) may also exist in their tautomeric forms. Such forms although not explicitly indicated in the above formula are intended to be included within the scope of the present invention.

Whenever used hereinafter, the term “compounds of formula (I)” or “the compounds according to the invention” is meant to include also the pharmaceutically acceptable acid or base addition salts and all stereoisomeric forms.

The compounds according to the invention can be prepared and formulated into pharmaceutical compositions by methods known in the art and in particular according to the methods described in the published patent specification mentioned herein and incorporated by reference; for the compounds of formula (I) suitable examples can be found in PCT publication WO-2004/105765. To prepare the aforementioned pharmaceutical compositions, a therapeutically effective amount of the particular compound, optionally in addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions (including nanosuspensions), syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions containing compounds of formula (I) may be formulated in an oil for prolonged action. Appropriate oils for this purpose are, for example, peanut oil, sesame oil, cottonseed oil, corn oil, soy bean oil, synthetic glycerol esters of long chain fatty acids and mixtures of these and other oils. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gels, dressings, shampoos, tinctures, pastes, ointments, salves, powders and the like. Application of said compositions may be by aerosol, e.g. with a propellent such as nitrogen, carbon dioxide, a freon, or without a propellent such as a pump spray, drops, lotions, or a semisolid such as a thickened composition which can be applied by a swab. In particular, semisolid compositions such as salves, creams, gels, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Preferably, a therapeutically effective amount of the pharmaceutical composition comprising a compound according to the invention, is administered orally or parenterally. Said therapeutically effective amount is the amount that effectively prevents metastasis and/or growth or reduces the size of a variety of neoplastic disorders or cell proliferative disorders (supra) in patients. On the basis of the current data, it appears that a pharmaceutical composition comprising a compound of the present invention, and in particular 4,6-ethanediylidenepyrimido [4,5-b][6,1,12]benzoxadiazacyclo-pentadecine, 17-bromo-8,9,10,11,12,13,14,19-octahydro-20-methoxy-13-methyl- as the active ingredient can be administered orally in an amount of from 10 mg to several (1 to 5) grams daily, either as a single dose or subdivided into more than one dose, including, e.g. two, three or even four times daily. A preferred amount ranges from 500 to 4,000 mg daily. A particularly, preferred dosage for such a compound is in the range of 750 mg to 3,000 mg daily. It will be appreciated that the amount of a compound according to the present invention, also referred to here as the active ingredient, which is required to achieve a therapeutic effect will, of course, vary with, the route of administration, the age and condition of the recipient, and the particular disorder or disease being treated. The optimum dosage amounts and regimen can be readily determined by those skilled in the art using conventional methods and in view of the information set out herein. This treatment can be given either continuously or intermittently, including, e.g., but not limited to, cycles of 3-4 weeks with treatment given for 1-21 days per cycle or other schedules shown to be efficacious and safe.

The above VEGFR3 inhibitors may be used in combination with one or more other cancer treatments. Such combinations could encompass any established antitumor therapy, such as, but not limited to, chemotherapies, irradiation, and target based therapies such as antibodies and small molecules. These therapies may be combined in systemic therapy, or local instillation/administration (e.g. intrathecally), depending on optimum efficacy/safety requirements.

In certain preferred embodiments, one or more other cancer treatments suitable in combination with the VEGFR3 inhibitors of the present invention, with respect to the different tumor types, include, but are not limited to;
breast: herceptin, docetaxel, anthracyclin, capecitabine
prostate: docetaxel, mitoxantrone
colon: oxaliplatin, 5-FU, avastin, irinotecan, cetuximab
lung; taxotere, carboplatin, gemicitabine

The VEGFR3 inhibitor of the invention and the further anti-cancer agent may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimens for each component of the combination will depend on the particular VEGFR3 inhibitor and further anti-cancer agents being administered, their route of administration, the particular tumor being treated and the particular host being treated. The optimum method and order of administration and the dosage amounts and regimen can be readily determined by those skilled in the art using conventional methods and in view of the information set out herein.

### PREPARATION OF COMPOUNDS AND FORMULATIONS

Preparation of Compound 95, 12H-4,6-ethanediylidene-13,17-methanopyrimido[4,5]-[b16,1,10,16]benzoxatriazacyclononadecin-12-one, 21-chloro-8,9,10,11,13,14,15,16,18,23-decahydro-25-methoxy-

Step 1.

Titanium, tetrakis(2-propanolato) (0.005 mol) was added to a solution of 3-piperidinecarboxylic acid ethyl ester (0.54 g, 0.005 mol) and 4-chloro-2-nitrobenzaldehyde (0.93 g, 0.005 mol) in CH2Cl2 (40 ml). The mixture was stirred for 1 hour at room temperature and NaBH(OAc)3 (0.0055 mol) was added. The reaction mixture was stirred for 1 hour at room temperature and extra NaBH(OAc)3 (0.00275 mol) was added and the mixture was stirred for 2 hours at room temperature and then NaHCO3 (saturated in H2O) was added. The organic layer was separated, dried (K2CO3), filtered and the solvent was evaporated and co-evaporated with toluene to remove the residual CH2Cl2. The 3-piperidinecarboxylic acid, 1-[(4-chloro-2-nitrophenyl)methyl]-, ethyl ester as crude residue was used as such in the next reaction step.

Step 2.

A mixture of 3-piperidinecarboxylic acid, 1-[(4-chloro-2-nitrophenyl)methyl]-, ethyl ester (1.69 g, 0.005 mol) in ethanol (150 ml) was hydrogenated with Pt/C 5% (0.5 g) as a catalyst in the presence of a thiophene solution (1 ml). After uptake of H2 (3 equiv.), the catalyst was filtered off and the filtrate was evaporated. The residue was redissolved in EtOAc and this solution was filtered over Extrelute. Heptane was added but no crystals appeared. The solvent was evaporated and the residue was redissolved in CH3OH and filtered over a paper filter to remove silica grease. The solvent was evaporated to yield 1.35 g of 3-piperidinecarboxylic acid, 1-[(2-amino-4-chlorophenyl)methyl]-, ethyl ester as a brown oil.

Step 3.

4-Chloro-7-methoxy-6-quinazolinol 6-acetate (0.273 g, 0.00108 mol) was added to a solution of 3-piperidinecarboxylic acid, 1-[(2-amino-4-chlorophenyl)methyl]-, ethyl ester (0.0011 mol) in 2-propanol (q.s.) and then the reaction mixture was shaken overnight at 80° C. Then the mixture was further shaken for 6.5 hours at 80° C. and the solvent was evaporated. Yield: 3-piperidinecarboxylic acid, 1-[[2-[[6-(acetyloxy)-7-methoxy-4-quinazolinyl]amino]-4-chlorophenyl]methyl]-, ethyl ester (crude; used as such in the next reaction step).

Step 4.

NH3/CH3OH 7N (approx. 10 ml) was added to 3-piperidinecarboxylic acid, 1-[[2-[[6-(acetyloxy)-7-methoxy-4-quinazolinyl]amino]-4-chlorophenyl]methyl]-, ethyl ester (0.00114 mol) and the reaction mixture was shaken for 1 hour at 35° C. and then the solvent was evaporated. Yield: 3-piperidinecarboxylic acid, 1-[[4-chloro-2-[(6-hydroxy-7-methoxy-4-quinazolinyl)amino]phenyl]methyl]-, ethyl ester (used as such in the next reaction step).

Step 5.

A mixture of 3-piperidinecarboxylic acid, 1-[[4-chloro-2-[(6-hydroxy-7-methoxy-4-quinazolinyl)amino]phenyl]methyl]-, ethyl ester (0.00114 mol), N-(3-bromopropyl)carbamic acid 1,1-dimethylethyl ester (1 eq) and Cs2CO3 (1.8582 g) was stirred overnight at room temperature and then the reaction mixture was stirred for 30 minutes at 50° C. When necessary more N-(3-bromopropyl)carbamic acid 1,1-dimethylethyl ester was added and the mixture was stirred for 4 hours at room temperature and for another 15 min. at 50° C. The solvent was evaporated (Genevac) and the residue was dissolved in CH2Cl2. This solution was filtered over dicalite and the filtrate was evaporated. Yield: 3-piperidinecarboxylic acid, 1-[[4-chloro-2-[[6-[3- [[(1,1-dimethylethoxy)carbonyl]amino]propoxy]-7-methoxy-4-quinazolinyl]amino]phenyl]methyl]-, ethyl ester.

Step 6.

A solution of 3-piperidinecarboxylic acid, 1-[[4-chloro-2-[[6-[3-[[(1,1-dimethylethoxy)carbonyl]amino]propoxy]-7-methoxy-4-quinazolinyl]amino]phenyl]methyl]-, ethyl ester (residue; approx 0.00114 mol) in TFA/CH2Cl2/TIS (90/8/2) (11.4 ml) was stirred for 7-8 hours, then the solvent was evaporated and the obtained residue was dried overnight in an oven. Yield: 3-piperidinecarboxylic acid, 1-[[2-[[6-(3-aminopropoxy)-7-methoxy-4-quinazolinyl]amino]-4-chlorophenyl]methyl]-.CF3COOH.

Step 7.

A solution of 3-piperidinecarboxylic acid, 1-[[2-[[6-(3-aminopropoxy)-7-methoxy-4-quinazolinyl]amino]-4-chlorophenyl]methyl]-.CF3COOH (0.00114 mol) and DIPEA (0.00684 mol) was added to a solution of HBTU (0.00342 mol) and HOBt (0.00228 mol) in dry DMF (285 ml) and then the reaction mixture was reacted for 1 hour. The solvent was evaporated and the dry residue was purified by reversed-phase high-performance liquid chromatography. The product fractions were collected, Na2CO3 was added and the organic solvent was evaporated. CH2Cl2 was added to the aqueous concentrate and the resulting mixture was extracted 3 times with CH2Cl2, then the organic extract was dried and collected. Yield: 0.0007 mol of 12H-4,6-ethanediylidene-13,17-methanopyrimido[4,5-b][6,1,10,16]benzoxatriazacyclononadecin-12-one, 21-chloro-8,9,10,11,13,14,15,16,18,23-decahydro-25-methoxy- (62% yield).

Preparation of Compound 96, 4,6-ethanediylidene-12H-pyrimido[4,5-b][6,1,10,13]benzoxatriazacvclohexadecin-12-one, 18-chloro-8,9,10,11,13,14,15,20-octahydro-21-methoxy-13,14-dimethyl-, (13S)-

Step 1.

A mixture of N-methyl-L-alanine methyl ester hydrochloride (1.4 g, 0.010 mol), 4-chloro-2-nitrobenzaldehyde (0.010 mol) and Titanium, tetrakis(2-propanolato) (0.010 mol) in CH2Cl2 (q.s.) was stirred for 1 hour at room temperature. Then NaBH(OAc)3 (0.022 mol) was added and the mixture was stirred for 3 hours at room temperature. More NaBH(OAc)3 (0.011 mol) was added and the mixture was stirred overnight at room temperature. Then NaHCO3 (saturated) was added till basic and the mixture was filtered over a P3 filter. The organic layer was separated and the water layer was extracted 3 times with CH2Cl2. The combined organic layers were dried (K2CO3) and the solvent was evaporated to dryness. The crude residue, L-alanine, N-[(4-chloro-2-nitrophenyl)methyl]-N-methyl-, methyl ester, was used as such in the next reaction step.

Step 2.

A mixture of L-alanine, N-[(4-chloro-2-nitrophenyl)methyl]-N-methyl-, methyl ester (1.03 g, 0.0036 mol) in CH3OH (50 ml) was hydrogenated with Pt/C 5% (0.5 g) as a catalyst in the presence of a 4% thiophene solution in DIPE (0.5 ml). After uptake of H2 (3 equiv.), the catalyst was filtered off and the filtrate was evaporated. The residue was purified over silica gel (with cartridge) (eluent: 4% Et3N / CH2Cl2). The desired fractions were collected. Heptane was added to the desired fraction (2 phases form). The heptane layer was separated to remove silica grease. After removal of the solvent and drying 0.5674 g of L-alanine, N-[(2-amino-4-chlorophenyl)methyl]-N-methyl-, methyl ester was obtained (22%; yellow oil). The crude product was used as such in the next reaction step.

Step 3.

4-Chloro-7-methoxy-6-quinazolinol 6-acetate (0.00055 mol) was added to a solution of L-alanine, N-[(2-amino-4-chlorophenyl)methyl]-N-methyl-, methyl ester (0.00055 mol) in 2-propanol (5 ml) and then the reaction mixture was shaken overnight at 80° C. and the solvent was evaporated. Yield: L-alanine, N-[[2-[[6-(acetyloxy)-7-methoxy-4-quinazolinyl]amino]-4-chlorophenyl]methyl]-N-methyl-, methyl ester (crude, used as such in the next reaction step).

Step 4.

NH3/CH3OH 7N (10 ml) was added to L-alanine, N-[[2-[[6-(acetyloxy)-7-methoxy-4-quinazolinyl]amino]-4-chlorophenyl]methyl]-N-methyl-, methyl ester (0.00055 mol) and the reaction mixture was shaken for 1-2 hours and then the solvent was evaporated. Yield: L-alanine, N-[[4-chloro-2-[(6-hydroxy-7-methoxy-4-quinazolinyl)amino]phenyl]methyl]-N-methyl-, methyl ester (used as such in the next reaction step).

Step 5.

A mixture of L-alanine, N-[[4-chloro-2-[(6-hydroxy-7-methoxy-4-quinazolinyl)amino]phenyl]methyl]-N-methyl-, methyl ester (0.00055 mol), N-(3-bromopropyl)carbamic acid 1,1-dimethylethyl ester (1 eq)and Cs2CO3 (0.8965 g) was stirred overnight at room temperature and then the reaction mixture was stirred for 30 minutes at 50° C. When necessary, more CAS N-(3-bromopropyl)carbamic acid 1,1-dimethylethyl ester was added and the mixture was stirred for 4 hours at room temperature and for another 15 minutes at 50° C. The solvent was evaporated (Genevac) and the residue was dissolved in CH2Cl2. This solution was filtered over dicalite and the filtrate was evaporated. Yield: L-alanine, N-[[4-chloro-2-[[6-[3-[[(1,1-dimethylethoxy)carbonyl]amino]propoxy]-7-methoxy-4-quinazolinyl]amino]phenyl]methyl]-N-methyl-, methyl ester.

Step 6.

A solution of L-alanine, N-[[4-chloro-2-[[6-[3-[[(1,1-dimethylethoxy)carbonyl]amino]propoxy]-7-methoxy-4-quinazolinyl]amino]phenyl]methyl]-N-methyl-, methyl ester (crude residue; 0.00055 mol) in TFA/CH2Cl2/TIS (90/8/2) (5.5 ml) was stirred for 7-8 hours, then the solvent was evaporated and the obtained residue was dried overnight in an oven. Yield: L-alanine, N-[[2-[[6-(3 -aminopropoxy)-7-methoxy-4-quinazolinyl]amino]-4-chlorophenyl]methyl]-N-methyl-.CF3COOH.

Step 7.

A solution of L-alanine, N-[[2-[[6-(3-aminopropoxy)-7-methoxy-4-quinazolinyl]amino]-4-chlorophenyl]methyl]-N-methyl-.CF3COOH (0.00055 mol) and DIPEA (0.0033 mol) was added to a solution of HBTU (0.00165 mol) and HOBt (0.0011 mol) in dry DMF (137 ml) and then the reaction mixture was reacted for 1 hour. The solvent was evaporated and the dry residue was purified by reversed-phase high-performance liquid chromatography. The product fractions were collected, Na2CO3 was added and the organic solvent was evaporated. CH2Cl2 was added to the aqueous concentrate and the resulting mixture was extracted 3 times with CH2Cl2, then the organic extract was dried and collected. Yield: 0.049 g of 4,6-ethanediylidene- 12H-pyrimido[4,5-b][6,1,10,13]benzoxatriazacyclohexadecin-12-one, 18-chloro-8,9,10,11,13,14,15,20-octahydro-21-methoxy-13,14-dimethyl-, (13S)-.

As used in the examples, ‘CH₃OH’ means methanol, ‘Et₃N’ means triethylamine, ‘CH₂Cl₂’ means dichloromethane, ‘HBTU’ means 1-[bis(dimethylamino)methylene]-1H-Benzotriazoliumhexafluorophosphate( 1-)3-oxide‘DMF’ means N,N-dimethylformamide, ‘NaBH(OAc)₃’ means sodium triacetoxyborohydride, ‘DIPEA’ means N-ethyl-N-(1-methylethyl)-2-propanamine, ‘HOBt’ means 1-hydroxy-1H-benzotriazole, ‘TFA’ means trifluoroacetic acid, ‘TIS’ means tris(1-methylethyl)silane, ‘K₂CO₃’ means potassium carbonate, ‘Cs₂CO₃’ means cesium carbonate, ‘Na₂CO₃’ means carbonic acid disodium salt, ‘NaHCO₃’ means carbonic acid monosodium salt.

Preparation of Compound 22, MTKI1

A suitable preparation of the preferred compound used in this invention, taken from WO-2004/105765, follows:

### EXAMPLE A

a) Preparation of 1-pentanol, 5-[[(4-bromo-2-nitrophenyl)methyl]amino]-(intermediate 1)

A solution of 4-bromo-2-nitro-benzaldehyde,(0.013 mol), 5-amino-1-pentanol (0.013 mol) and titanium, tetrakis (2-propanolato) (0.014 mol) in EtOH (15 ml) was stirred at RT for 1 hour, then the reaction mixture was heated to 50° C. and stirred for 30 min. The mixture was cooled to RT and NaBH₄ (0.013 mol) was added portionwise. The reaction mixture was stirred overnight and then poured out into ice water (50 ml). The resulting mixture was stirred for 20 min., the formed precipitate was filtered off (giving Filtrate (I)), washed with H₂O and stirred in DCM (to dissolve the product and to remove it from the Ti-salt). The mixture was filtered and then the filtrate was dried (MgSO₄) and filtered, finally the solvent was evaporated. Filtrate (I) was evaporated until EtOH was removed and the aqueous concentrate was extracted 2 times with DCM. The organic layer was separated, dried (MgSO₄), filtered off and the solvent was evaporated, yielding 3.8 g (93%) of intermediate 1.

### EXAMPLE B

a) Preparation of 1-pentanol, 5-[[(4-bromo-2-nitrophenyl)methyl]methylamino]-(intermediate 2)

A solution of intermediate 50 (0.0047 mol), formaldehyde (0.025 mol) and titanium, tetrakis (2-propanolato) (0.005 1 mol) in EtOH (150 ml) was heated to 50° C. and stirred for 1 hour, then NaBH₄ (0.026 mol) was added portionwise at RT. The reaction mixture was stirred overnight and then quenched with water (100 ml). The resulting mixture was stirred for 1 hour; the formed precipitate was filtered off and washed. The organic filtrate was concentrated, then the aqueous concentrate was extracted with DCM and dried. The solvent was evaporated and the residue was filtered over silica gel (eluent: DCM/CH₃OH from 98/2 to 95/5). The product fractions were collected and the solvent was evaporated, yielding 0.5 g of intermediate 2.

b) Preparation of 1-pentanol, 5-[[(4-bromo-2-nitrophenyl)methyl]methylamino]-, acetate (ester) (intermediate 3)

A solution of intermediate 2 (0.0015 mol) and pyridine (0.015 mol) in acetic anhydride (8 ml) was stirred overnight at RT, then the solvent was evaporated and co-evaporated with toluene, yielding intermediate 3.

c) Preparation of 1-pentanol, 5-[[(2-amino-4-bromophenyl)methyl]methylamino]-, acetate (ester) (intermediate 4)

A mixture of intermediate 3 (0.0015 mol) in THF (50 ml) was hydrogenated with Pt/C 5% (0.5 g) as a catalyst in the presence of thiophene solution (0.5 ml) [H179-034]. After uptake of H₂ (3 equiv.), the catalyst was filtered off and the filtrate was evaporated, yielding 0.5 g of intermediate 4.

d) Preparation of 6-quinazolinol, 4-[[2-[[[5-(acetyloxy)pentyl]methylamino]methyl]-5-bromophenyl]amino]-7-methoxy-, acetate (ester) (intermediate 5)

A mixture of intermediate 4 (0.0015 mol) and 4-chloro-7-methoxy-6-quinazolinol acetate (ester) (0.0015 mol) in 2-propanol (30 ml) was heated to 80° C. and the reaction mixture was stirred for 1 day. The solvent was evaporated under reduced pressure and the residue was used as such in the next reaction step, yielding 0.83 g of intermediate 5.

e) Preparation of 6-quinazolinol, 4-[[5-bromo-2-[[(5-hydroxypentyl)methylamino]methyl]phenyl]amino]-7-methoxy-(intermediate 6)

A solution of intermediate 5 (0.0015 mol) in methanol (25 ml) was stirred at RT and a solution of K₂CO₃ (0.003 mol) in H₂O (2.5 ml) was added, then the reaction mixture was heated to 60° C. and stirred for 18 hours. The solvent was evaporated and H₂O (20 ml) was added, then the mixture was neutralized with acetic acid and the formed precipitate was filtered off. The filtrate was concentrated under reduced pressure and the concentrate was extracted with DCM, filtered, then dried (MgSO₄) and the mixture was concentrated under reduced pressure, yielding 0.5 g (70%) of intermediate 6.

### EXAMPLE C

a) Preparation of 4,6-ethanediylidenepyrimido[4,5-b][6,1,12]benzoxadiazacyclo-pentadecine, 17-bromo-8,9,10,11,12,13,14,19-octahydro-20-methoxy-13-methyl-(compound MTKI1)

A solution of intermediate 6 (0.0011 mol) in THF (50 ml) was stirred at RT and tributylphosphine (0.0016 mol) was added, then 1,1′-(azodicarbonyl)bis-piperidine (0.0016 mol) was added and the reaction mixture was stirred for 2 hours. The solvent was evaporated until ⅓ of the initial volume. The resulting precipitate was filtered off and washed. The filtrate was evaporated and the residue was purified by RP high-performance liquid chromatography. The product fractions were collected and the organic solvent was evaporated. The aqueous concentrate was extracted 2 times with DCM and the organic layer was dried (MgSO₄), then filtered off. The solvent was evaporated and the residue was dried (vac.) at 50° C., yielding 0.004 g (0.8%) of compound MTKI1.

Preparation of Compound 2,4,6-ethanediylidene-19-H-pyrimido[4,5-b][16,13,1]benzodioxaazacyclo-pentadecine, 15-chloro-8,9,10,11,12,13-hexahydro-20-methoxy-;

### EXAMPLE D

Preparation of 4,6-ethanediylidene-19H-pyrimido[4,5-b][6,13,1]benzodioxaazacyclo-pentadecine, 15-chloro-8,9,10,11,12,13- hexahydro-20-methoxy- (compound 2)

A solution of intermediate 9 (0.0024 mol) and triphenylphosphine (0.0036 mol) in THF, dry (100 ml) was stirred at RT and then a solution of bis(1-methylethyl)diazenedicarboxylate (0.0036 mol) in THF (10 ml) was added dropwise. The reaction mixture was stirred for 6 hours and extra bis(1-methylethyl)diazenedicarboxylate (0.35 ml) in THF (10 ml) was added. The mixture was stirred overnight and concentrated. The residue was purified by column chromatography over silica gel (eluent: DCM/CH₃OH/THF 90/5/5). The product fractions were collected and further purified by RP high-performance liquid chromatography. The product fractions were collected and concentrated. The aqueous concentrate was filtered, and the solid retained washed and dried (vac.) at 65° C., yielding 0.065 g of compound 2, melting point 255.5-260.2° C.

### EXAMPLE E

a) Preparation of hexanoic acid, 6-(2-chloro-6-nitrophenoxy)-, methyl ester (intermediate 6)

A solution of 2-chloro-6-nitro-phenol (0.046 mol) in N,N-dimethylformamide (150 ml) was heated to 50° C., then K₂CO₃ (0.069 mol) was added and the reaction mixture was stirred for 15 min. 6-Bromo-, methyl ester hexanoic acid (0.069 mol) was added and the mixture was stirred overnight. The reaction mixture was filtered and the filtrate was concentrated and the residue was used as such in the next step, yielding 13.88 g of intermediate 6.

b) Preparation of hexanoic acid, 6-(2-amino-6-chlorophenoxy)-, methyl ester (intermediate 7)

A mixture of intermediate 6 (0.046 mol) and ethanime (2 g) in THF (ml) was hydrogenated with Pt/C 5% (3 g) as a catalyst in the presence of DIPE (2 ml). After uptake of H₂ (3 equiv.), the reaction mixture was filtered over small plug of Dicalite the filtrate was concentrated, yielding intermediate 7.

c) Preparation of hexanoic acid, 6-[2-[[6-(acetyloxy)-7-methoxy-4-quinazolinyl]amino]-6-chlorophenoxy]-, methyl ester (intermediate 8)

A mixture of 4-chloro-6-methylcarbonyloxy-7-methoxyquinazoline (0.022 mol) and intermediate 7 (0.022 mol) in 2-propanol (170 ml) was stirred and heated at 80° C. for 2 hours, concentrated and the residue was chromatographed over silica gel (eluent: DCMNCH₃OH 97/3). The product fractions were collected and the solvent was evaporated, yielding 5.1 g intermediate 8 (used as such in the next reaction step).

d) Preparation of 6-quinazolinol, 4-[[3-chloro-2-[(6-hydroxyhexyl)oxy]phenyl]amino]-7-methoxy-(intermediate 9)

A mixture of LAH (0.0246 mol) in THF (40 ml) was stirred at RT. A solution of intermediate 8 (0.006 mol) in THF (60 ml) was added dropwise. The reaction mixture was stirred for 1 day then, extra LAH (0.0123 mol) was added portionwise. The mixture was stirred further over the weekend then, H₂O (2 ml) was added dropwise, followed by the dropwise addition of a 15% NaOH soln. (2 ml) and H₂O (6 ml). This mixture was stirred for 15 min filtered and the filtrate was concentrated The residue was stirred in boiling CH₃CN, filtered and the solid retained was dried (vac.) at 60° C. The solids were re-dissolved in CH₃OH/DCM (10/90) and this mixture was neutralised with HCl (1N). The organic layer was separated, dried (MgSO₄), filtered and concentrated, yielding 1 g of intermediate 9.

An illustrative formulation example for the most preferred compound, MYKI1, is as follows:

### EXAMPLE F

### Formulation

The product MTKI1 can be prepared as a 10-mg/mL oral solution, pH 2. It contains an excipient, Captisol® (chemical name: sulfobutyl ether-β-cyclodextrin, SBE-β-CD), citric acid, Tween® 20, HCl, and NaOH in purified water. The formulation can be stored refrigerated (2-8° C.; 36-46° F.) and allowed to warm to room temperature for maximally 1 hour prior to dose preparation.

The product MTKI1 can also be prepared as 50-mg, 100-mg and 300-mg oral immediate release capsules, containing the active chemical entity MTKI1, lactose monohydrate (200 mesh), sodium lauryl sulphate and magnesium stearate in hard gelatin capsules, sizes 3, 4 and 00, respectively. The capsules may also contain any or all of the following ingredients: gelatin, red iron oxide and titanium oxide.

### EXPERIMENTAL DATA

VEGFR3 Inhibition

Lymph node involvement is a poor prognostic factor in a number of cancer types. Recent studies have demonstrated that lymphatic vessel formation plays an important role in tumor progression and vascular endothelial growth factor (VEGF) C and D have been identified as specific lymphangiogenic factors that act via activation of the cognate receptor VEGFR3. 4,6-ethanediylidenepyrimido[4,5-b][6,1,12]benzoxadiazacyclo-pentadecine, 17-bromo-8,9,10,11,12,13,14,19-octahydro-20-methoxy-13-methyl- , hereinafter also referred to as Compound 2, is a multitargeted kinase inhibitor that has been shown to have a unique kinase inhibition profile (pan Her and Src family) as well as a favourable tissue distribution profile resulting in potent anti tumor activity in a number of experimental models.

In vitro kinase assays have shown that the compounds of the present invention have potent inhibition of the EGFR, Her2, Her4 and Src family kinases (Src, Fyn, Lck, Yes, Lyn) activity.

The in vitro kinase inhibition was validated herein in cell based assays using human vascular endothelial cells (HMVECd), in which Compound 2 prevented VEGFR3 dependent VEGF-C stimulation of Erk1/2, whereas it did not affect VEGFR1 mediated VEGF signaling. Compound 2 has also been shown herein to inhibit VEGF-C induced phosphoylation of VEGFR3 using a cell line engineered to overexpress human VEGFR3.

These results were further validated herein in a recently developed Xenopus tadpole model where Compound 2 was found to phenocopy the effects of VEGF-C knockout. (Ny et al, Nat. Med. 2005 September; 11:998-1004).

### Methods

In Vitro Kinase Assays

The VEGFR3 kinase reaction was performed at 30° C. for 10 minutes in a 96-well microtiterplate. The 25 μl reaction volume contained 8 mM MOPS pH 7, 200 μM EDTA, 10 mM MgAc, 10 μM unlabeled ATP, 0.5 mCi AT33P, 500 μM JAK3-tide (Ac-GGEEEEYFELVKKKK-NH2), 25 ng VEGFR3 and 2% compound in 100% DMSO.

The reaction was stopped by adding 5 μl of a 3% phosphoric acid solution. 10 μl of the reaction mixture was then spotted onto a Filtermat P30 filter (Wallac) and washed 3 times for 5 min. in 0.75% phosphoric acid and 1 time for 2 min. in methanol prior to transferring to a sealable plastic bag containing 4 ml of scintillation liquid and reading in a scintillation counter.

### VEGFR3 Cellular Assays

Inhibition of VEGF-C Induced VEGFR3 Activity

For this assay, porcine aortic endothelial (PAE) cells stably expressing human VEGFR-3 were grown to confluency on 10 cm dishes. The cells were serum starved overnight and the media replaced with fresh serum-free media. Pre-determined amounts of Compound 2 or control vehicle was added to the cells, and then incubated at 37° C. for 15 min. This was followed by the addition of VEGF-C to a final concentration of 100 ng/ml and the cells incubated for 10 min at 37° C., after which they were washed with ice-cold PBS and then lysed. Cell lysates containing protease inhibitors were subjected to immunoprecipitation with an antibody specific for VEGFR-3 and the immunoprecipitates separated by SDS-PAGE. Proteins were transferred to a nitrocellulose membrane, and then probed first with a phospho-tyrosine antibody and then a VEGFR-3 antibody.

### Inhibition of Erk Activation Induced by VEGF-C in Human Micro Vessel Endothelial Cells-Dermal (HMVEC-d)

HMVEC-d cells (Cambrex) were cultured at 37 C in 5% CO2 in appropriate serum containing medium (Cambrex) on fibronectin coated 12-well plates (BD Biocoat, Becton Dickinson) for 4 days. Cells were then starved for 16 hours in the same medium as before, but lacking serum and containing SITE+3 (Invitrogen) instead. Cells were then preincubated with DMSO at 0.2% (the solvent) or 5 uM Compound 2 for 60 minutes and then either 10 ng/ml VEGF or 100 ng/ml VEGF-C (R&D systems) was added to the cells. Before, or 10 and 30 minutes after stimulation, the medium was removed, cells were rinsed with ice-cold PBS containing 0.1 mM sodiumorthovanadate and lysed in M-PER buffer (PIERCE) containing 0.1% SDS, and 1× phosphatase and protease inhibitors (HALT, PIERCE). Cells were scraped off the plates and incubated in lysis buffer for 30 minutes on ice and then centrifuged at 4° C. for 10 min at 16000×g. Lysates were quantified using the BCA reagent according to the manufacturer's instructions (PIERCE), and 5 mg of total protein was loaded on SDS-polyacrylamide gels (NUPAGE, Invitrogen). Electrophoresis and blotting onto PVDF membranes was all performed using reagents from the NUPAGE system, according to the manufacturer's instructions. Blots were blocked for 1 hour with Odyssey blocking buffer (LICOR) and then primary antibodies (see below) were diluted 1:1000 in a 1:1 mix of Odyssey blocking buffer and PBS containing 0.1% Tween-20, and applied overnight at 4° C.: P-Erk1/2 (Thr202/Tyr204, clone E10, mouse monoclonal #9106, Erk1/2 (rabbit polyclonal #9102) (all from Cell Signaling Technologies, Inc). Blots were washed three times in PBS containing 0.1% Tween for 5 min and then incubated for 1 hour with secondary antibodies (diluted 1:1000 in PBS containing 0.1% Tween-20):goat anti-mouse conjugated to IRDye 800 nm (Rockland, Inc) or goat anti-rabbit conjugated to Alexa 680 nm (Molecular Probes, Inc).

Blots were washed three times in PBS containing 0.1% Tween for 5 min and once in PBS and scanned using the LICOR system at 700 nm and 800 nm using intensity 5 and all settings appropriate for the scanning of membranes.

### VEGFR3 in Vivo Xenopus Assay

Xenopus tadpoles were used as a model (Ny et al Nature Medicine 11: 998-1004 (2005)) to analyze the effects of Compound 2 on lymphangiogenesis. The compound was administered daily in a dose dependent manner, with vehicle controls, to the medium of the Xenopus tadpoles starting at stage 26-28, i.e., before the formation of both blood and lymphatic vessels. The effects of the compound was monitored by “live analysis”, i.e. live embryos were examined 4 days later for evidence of lymph-vascular defects by trained observers. As an additional means of confirming the results, in a limited number of experiments, tadpoles were fixed at stage 35-36 and in situ hybridizations for Prox1 were performed to assess migration of lymphatic endothelial cells (LECs) from the posterior cardinal vein (PCV) to their dorsal endpoint, this reflecting an essential step in lymphangiogenesis. Under normal circumstances, LECs arise from the region of the PCV, accumulate there, and during the study period, they migrate dorsally to defined “areas” (Area 1 to Area 2 and Area 3), where they then further migrate rostrally and caudally to form the lymphatic vasculature in the tail and trunk. Defects in migration of the LECs from the PCV was quantified by determining the maximal migration of the cells, and by counting the number of cells in these different regions in the tail.

### Results

In Vitro Kinase Assays

The following table provides the pIC50 values obtained for the compounds of the present invention using the above-mentioned VEGFR3 kinase assay.

| Structure | Name | pic50 |
|---|---|---|
| 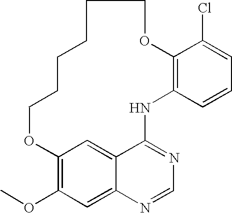 | 4,6-ethanediylidene-19H-pyrimido[4,5-b][6,13,1]benzodioxaazacyclo-pentadecine, 15-chloro-8,9,10,11,12,13-hexahydro-20-methoxy- | 6 |
| 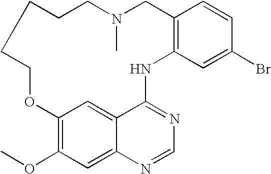 | 4,6-ethanediylidenepyrimido[4,5-b][6,1,12]benzoxadiazacyclopenta-decine, 17-bromo-8,9,10,11,12,13,14,19-octahydro-20-methoxy-13-methyl- | 7.74 |
| 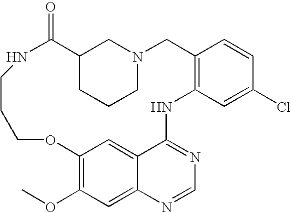 | 12H-4,6-ethanediylidene-13,17-methanopyrimido[4,5-b][6,1,10,16]benzoxatriazacyclo-nonadecin-12-one, 21-chloro-8,9,10,11,13,14,15,16,18,23-decahydro-25-methoxy- | 6 |
| 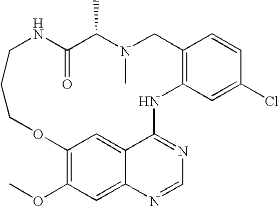 | 4,6-ethanediylidene-12H-pyrimido[4,5-b][6,1,10,13]benzoxatriazacyclo-hexadecin-12-one, 18-chloro-8,9,10,11,13,14,15,20-octahydro-21-methoxy-13,14-dimethyl- | 6 |

### Inhibition of VEGF-C Induced VEGFR-3 Phosphorylation in VEGFR-3 Expressing Porcine Aortic Endothelial Cells

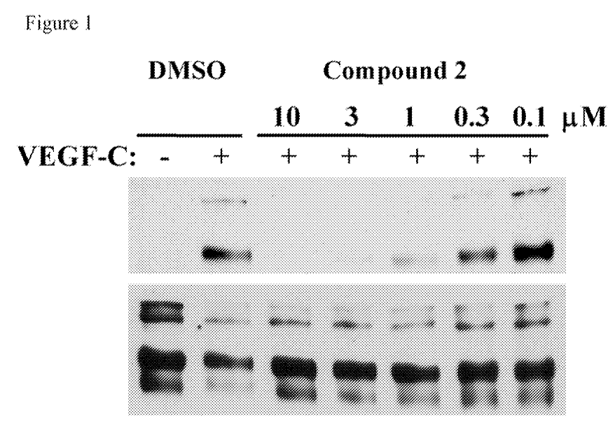

The effect of 4,6-ethanediylidenepyrimido[4,5-b][6,1,12]-benzoxadiazacyclo-pentadecine, 17-bromo-8,9,10,11,12,13,14,19-octahydro-20-methoxy-13-methyl-(Compound 2) on VEGF-C induced VEGFR3 activity is evident from the dose dependent reduction in VEGFR-3 phosphorylation (Top frame of FIG. 1). Presence of VEGFR-3 at the different concentration is confirmed upon re-staining of the Western Blots with VEGFR-3 antibodies (Bottom frame of FIG. 1).

### Erk Phosphorylation Assays in HMVEC-d Cells

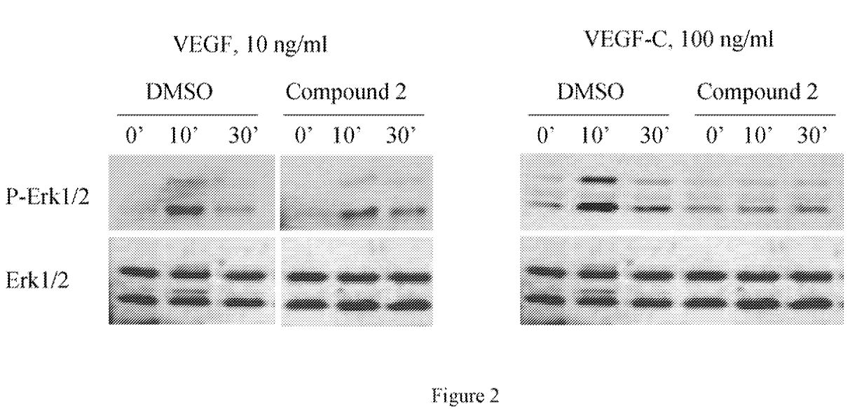

As illustrated in FIG. 2, Compound 2 at 5 μM, prevented phosphorylation of Erk1/2 by VEGF-C but did not interfere with VEGF stimulation of Erk1/2. These data are consistent with a selective inhibition of VEGFR-3 by Compound 2.

### Xenopus Study Results

The effects of Compound 2 was assessed using live screening, concentrations of Compound 2 of >50 uM induced hemorrhages in 30% of tadpoles. At 20 uM, 10% of tadpoles developed edema, which was not seen in vehicle-alone treated tadpoles. Compound 2 dose dependently inhibits Lymphatic Endothelial Cell (LEC) Migration in tadpoles (Table 1). LEC migration at stage 35-36 was particularly decreased, even at the lowest concentrations of compound tested (0.31 uM), and this was more than observed with vehicle alone. These data supports the conclusion that this compound interferes with normal lymphangiogenesis.

TABLE 1
Quantification of migration of lymphatic endothelial cells
(detected by Prox1 in situ hybridization) from posterior cardinal
vein towards dorsum after exposure of stages 35-36 tadpoles
to compounds (n = 10-15 embryos per condition)

| exp. | | | | | | |
|---|---|---|---|---|---|---|
| Aug. 08, | Concentration of Compound 2 in μM | | | | | |
| 2006 | 80 | 20 | 5 | 1.25 | 0.3125 | DMSO |
| % embryos | 80 | 60 | 40 | 63 | 57 | 21 |
| with | | | | | | |
| reduced | | | | | | |
| migration | | | | | | |
| % Relative | 56 ± 15 | 19 ± 8 | 123 ± 32 | 20 ± 8 | 37 ± 20 | |
| Area 2-3 | | | | | | |
| % Relative | 67 ± 21 | 35 ± 1 | 121 ± 3 | 47 ± 5 | 73 ± 22 | |
| Max | | | | | | |
| Migration | | | | | | |

Note:
The above assessments of LEC migration of Prox1 positive cells were first performed microscopically in a semi-quantitative manner by a trained observer (‘% embryos with reduced migration’). Computer-generated quantitative analysis were then performed. The “Relative Area 2-3” is the number of LECs that migrated to area 2-3 relative to the control (vehicle alone). “Relative max migration” is the furthest LEC migration from the PCV under treatment, relative to the max observed in the vehicle-alone controls.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method for the treatment or prevention of a VEGFR3 mediated biological activity in a mammalian subject comprising administering a therapeutically effective amount of a compound of formula (I) 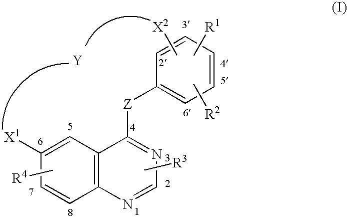
the pharmaceutically acceptable acid or base addition salts and the stereochemically isomeric forms thereof, wherein
Z represents NH;
Y represents —C₃₋₉alkyl-, —C₁₋₅alkyl-NR¹³—C₁₋₅alkyl-, —C₁₋₅alkyl-NR¹⁴—CO—C₁₋₅alkyl-, —C₁₋₃alkyl-NH—CO-Het²⁰-, or -Het²²-CH₂—CO—NH—C₁₋₃alkyl-;
X¹ represents O, or —O—C₁₋₂alkyl-;
X² represents a direct bond, —C₁₋₂alkyl-, O, —O—C₁₋₂alkyl-, NR¹² or NR¹²C₁₋₂alkyl-;
R¹ represents hydrogen, cyano, halo or hydroxy;
R² represents hydrogen, cyano, halo, or hydroxy;
R³ represents hydrogen;
R⁴ represents C₁₋₄alkyloxy-;
R¹² represents hydrogen, or C₁₋₄alkyl-;
R¹³ represents hydrogen or C₁₋₄alkyl;
R¹⁴ represents hydrogen or C₁₋₄alkyl;
Het²⁰ represents pyrrolidinyl, piperazinyl or piperidinyl; and
Het²² represents pyrrolidinyl, piperazinyl or piperidinyl, to a mammalian subject in need of such treatment.

2. The method as claimed in claim 1 wherein in the compound of formula (I);
Z represents NH;
Y represents —C₃₋₉alkyl-, —C₁₋₅alkyl-NR¹⁴—CO—C₁₋₅alkyl-, or —C₁₋₃alkyl-NH—CO-Het²⁰;
X¹ represents O;
X² represents —C₁₋₂alkyl-, O, or NR¹²—C₁₋₂alkyl-;
R¹ represents hydrogen or halo;
R² represents hydrogen or halo;
R³ represents hydrogen;
R⁴ represents C₁₋₄alkoxy;
R¹² represents C₁₋₄alkyl-;
R¹⁴ represents hydrogen or C₁₋₄alkyl; and
Het²⁰ represents pyrrolidinyl, piperazinyl or piperidinyl.

3. The method of claim 1, wherein the compound of formula (I) is selected from the group consisting of
4,6-ethanediylidene-19H-pyrimido[4,5-b][6,13,1]benzodioxaazacyclo-pentadecine, 15-chloro-8,9,10,11,12,13- hexahydro-20-methoxy-;
12H-4,6-ethanediylidene-13,17-methanopyrimido[4,5-b][6,1,10,16]benzoxatriazacyclononadecin-12-one, 21-chloro-8,9,10,11,13,14,15,16,18,23-decahydro-25-methoxy-;
4,6-ethanediylidene-12H-pyrimido[4,5-b][6,1,10,13]benzoxatriazacyclohexadecin-12-one, 18-chloro-8,9,10,11,13,14,15,20-octahydro-21-methoxy-13,14-dimethyl-; and
4,6-ethanediylidenepyrimido [4,5-b][6,1,12]benzoxadiazacyclopentadecine, 17-bromo-8,9,10,11,12,13,14,19-octahydro-20-methoxy-13-methyl-;
or a pharmaceutically acceptable acid addition salt thereof.

4. The method as claimed in claim 3, wherein the compound is
4,6-ethanediylidenepyrimido[4,5-b][6,1,12]benzoxadiazacyclo-pentadecine, 17-bromo-8,9,10,11,12,13,14,19-octahydro-20-methoxy-13-methyl-;
or a pharmaceutically acceptable acid addition salt thereof.

5. The method as claimed in claim 1, wherein a therapeutically effective amount of the compound is administered orally or parenterally.

6. The method as claimed in claim 1 wherein the compound of formula (I) is administered in combination with a further anti-cancer agent.

7. The method as claimed in claim 6, wherein the further anti-cancer agent is selected from the group consisting of herceptin, docetaxel, anthracyclin and capecitabine in case of breast cancer; docetaxel and mitoxantrone in case of prostate cancer; oxaliplatin, 5-FU, avastin, irinotecan and cetuximab in case of colon cancer; and taxotere, carboplatin and gemicitabine in case of lung cancer.

8. The method as claimed in claim 1, wherein the VEGFR3 mediated biological activity is selected from the group consisting of metastatic spread of a cancer in a mammalian subject; metastasis to regional lymph nodes via lymphatic vessels; tumor associated lymphangiogenesis in cancers; recruitment and proliferation of endothelial cells that express VEGFR3 in neovascularization of cancer cells that express a VEGFR3 ligand; and combinations thereof.

9. The method as claimed in claim 8, wherein said VEGFR3 ligand is VEGF-C, VEGF-D, or combination thereof.

10. The method of claim 1 in which the VEGFR3 mediated biological activity is advanced breast cancer in a mammal comprising the steps of administering a therapeutically effective amount of a compound as defined in any one of claims 1 to 4 to said mammal.
